(19) United States
(12) Plant Patent Application Publication
(10) Pub. No.: US 2002/0078483 P1
(43) Pub. Date: Jun. 20, 2002

(54) CORNUS ALBA PLANT NAMED 'CREAM CRACKER'

(76) Inventor: John Pannebakker, Hazerswoude (NL)

Correspondence Address: Christie, Parker & Hale, LLP, P.O. Box 7068, Pasadena, CA, 91109-7068, US

(21) Appl. No.: 09/738,151

(22) Filed: Dec. 15, 2000

(51) Int. Cl. A01H 5/00

(52) U.S. Cl. PLT/220

(57) ABSTRACT

A Cornus Alba plant variety producing attractive grey green foliage with cream-colored edges.

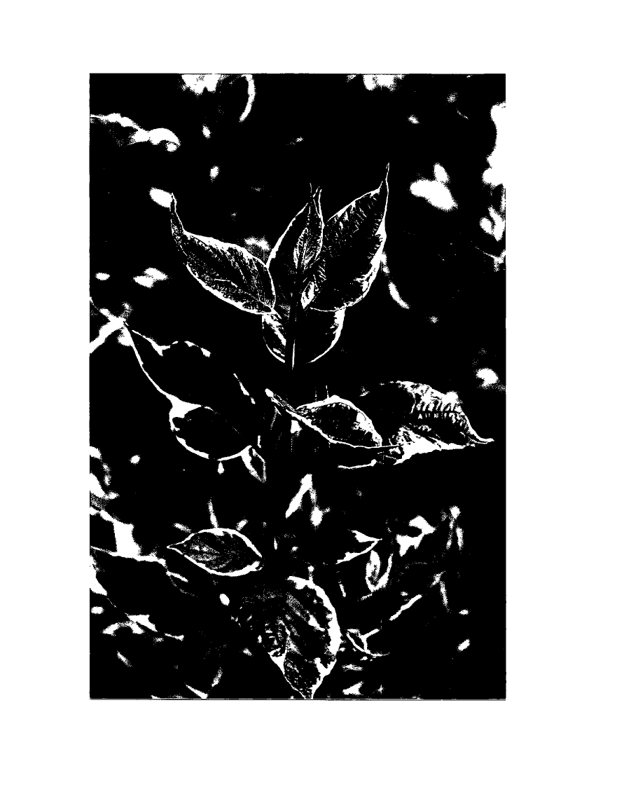

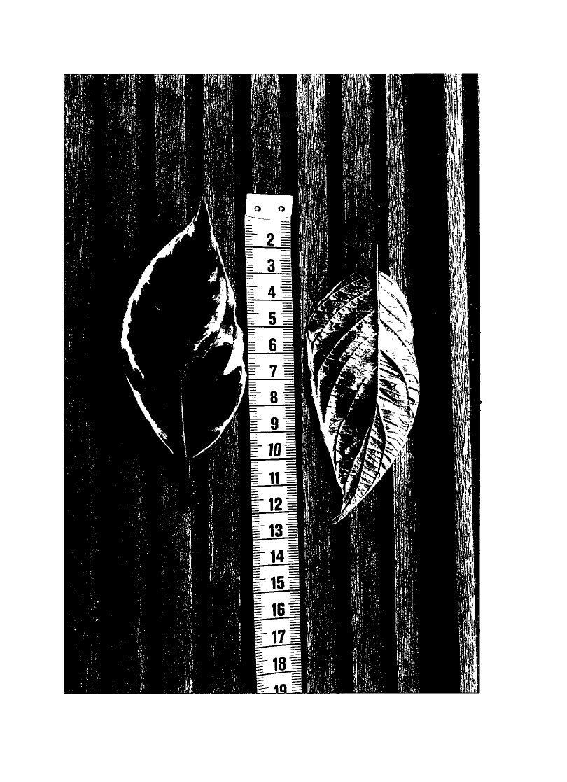

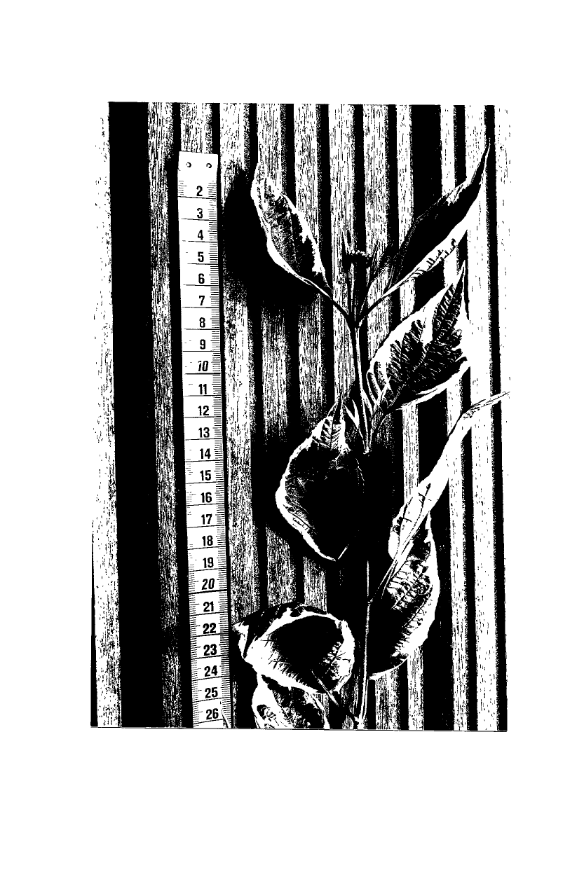

### BACKGROUND OF THE INVENTION

The present invention relates to a new and distinct Cornus Alba plant variety which was discovered by me in a controlled planting of Cornus Alba ‘Gouchaulbii’. The varietal denomination of this new variety is ‘Cream Cracker’.

### SUMMARY OF THE INVENTION

Among the novel characteristics possessed by the new variety which distinguish it from its parents and all other varieties of which I am aware are its attractive foliage.

Asexual reproduction by vegetative cuttings of the new variety as performed in Hazerswoude, The Netherlands, shows that the foregoing and other distinguishing characteristics come true to form and are established and transmitted through succeeding propagations. Other known forms of asexual reproduction may be also used.

### COMPARISON WITH PARENT

The new variety produces leaves with cream colored edges whereas the parent produces leaves with golden yellow color. The inside of leaves of ‘Cream Cracker’ are grey-green in contrast to merely green color of the parent. The overall appearance of the new variety is more vivid in contrast to the duller impression given by the parent.

### BRIEF DESCRIPTION OF ILLUSTRATION

The accompanying illustration shows a typical specimen of the vegetative growth and flowers of the new variety in different stages of development and depicted in color as nearly true as it is reasonably possible to make the same in a color illustration of this character.

### DESCRIPTION OF THE NEW VARIETY

The following is a detailed description of the new variety, from specimens grown under glass in Hazerswoude, The Netherlands. The color terminology used is in accordance with The Royal Horticultural Society Colour Chart (RHSCC), and refers to plate numbers in the aforementioned color chart. Phenotypic expression may vary depending on light, environmental and cultural conditions.

Plant:

Form.—Upright.

Growth habit and rate.—Vigorous.

Height (soil level to top of leaves).—About 2.5 m.

Foliage:

Leaf:

Shape.—Broad elliptic.

Quantity.—Densely covered.

Size.—About 8-11 cm.

Edge.—Smooth.

Texture.—Upper side is slightly hairy and underside is smooth.

Venation pattern.—Extends as bands, about six pairs, from the base.

Color:

Young foliage:

Upper side.—Inside light green, edges butter yellow with a touch of pink.

Underside.—Same as upper side only slightly paler.

Mature foliage:

Upper side.—Variegated green-grey with cream colored edges.

Underside.—Same as upper side but slightly more greyish.

Petiole.—Top surface is reddish and the underside is green and slightly reddish.

Flower stem length.—About 2-3 cm average.

Wood:

New shoots:

Color.—Greenish.

Bark.—Smooth.

Mature wood:

Color.—Purplish-red.

Bark.—Smooth.

Resistance to disease.—No susceptibility observed.

Stamens, Filaments and Anthers:

Arrangement.—Irregularly arranged around style.

Stamens:

Color.—White.

Anther:

Color.—Creamy white to brown.

Filament:

Color.—White.

Pollen:

Color.—Yellow.

Styles Uneven length:

Stigmas:

Color.—White.

Hips:

Shape.—Globular, seeds do not protrude.

Size.—About 2-4 mm.

Color.—White.

I claim:

1. A new and distinct Cornus Alba plant of the variety substantially as shown and described.
